SEQUENCE LISTING (4 sequences)

SEQ ID NO: 1 - DNA, 20 bp, Artificial
  Forward primer
gaacagccacacctacaaac

SEQ ID NO: 2 - DNA, 20 bp, Artificial
  Reverse primer
aaccctgctggtacatcatg

SEQ ID NO: 3 - DNA, 22 bp, Artificial
  Forward primer
tagcgaaaccagtgctggaaag

SEQ ID NO: 4 - DNA, 20 bp, Artificial
  Reverse primer
gcttgagggctcaaaaacag

(19) United States
(12) Patent Application Publication
(10) Pub. No.: US 2017/0265411 A1
(43) Pub. Date: Sep. 21, 2017

(54) PETUNIA FLOWER WITH NOVEL PIGMENTATION PATTERN

(71) Applicant: SYNGENTA PARTICIPATIONS AG, Basel (NL)

(72) Inventors: Dick Van Kleinwee, Enkhuizen (NL); Jolanda Krassenburg, Enkhuizen (NL); Sabine Lorente Rodriguez, Enkhuizen (NL)

(73) Assignee: SYNGENTA PARTICIPATIONS AG, Basel (NL)

(21) Appl. No.: 15/036,435

(22) PCT Filed: Nov. 17, 2014
(86) PCT No.: PCT/EP2014/074767

§ 371 Date: May 13, 2016

(30) Foreign Application Priority Data
Nov. 18, 2013 (EP) 13193320.2

(51) Int. Cl. A01H 5/02; A01H 1/02

(52) U.S. Cl. CPC A01H 5/02; A01H 1/02

(57) ABSTRACT

The present invention provides a Petunia flower having a novel pigmentation phenotype. Petunia plants and methods for making same are also provided.

### FIELD OF THE INVENTION

This invention relates to plants of the species Petunia hybrida. In particular, it relates to Petunia hybrida having a novel pigmentation phenotype.

### INTRODUCTION

Petunia is a genus of about 40 species of flowering plants of South American origin, closely related to tobacco, cape gooseberries, tomatoes, deadly nightshades, potatoes and chili peppers in the family Solanaceae. The popular flower of the same name derived its epithet from the French, which took the word petun, meaning “tobacco,” from a Tupi-Guarani language. An annual, most of the varieties seen in gardens are hybrids (Petunia×hybrida). The origin of P. hybrida is thought to be by hybridisation between P. axillaris (the night-scented petunia) and P. integrifolia (the violet-flowered petunia).

Many species of Petunia are known which include, but not limited to, P. alpicola, P. altiplana, P. axillaris, P. bajeensis, P. bonjardinensis, P. exserta, P. guarapuavensis, P. helianthemoides, P. humifusa, P. inflata, P. integrifolia, P. interior, P. ledifolia, P. littoralis, P. mantiqueirensis, P. occidentalis, P. parviflora, P. patagonica, P. pubescens, P. reitzii, P. riograndensis, P. saxicola, P. scheideana, P. variabilis, P. villadiana.

Petunia seeds typically germinate in 5 to 15 days and can tolerate relatively harsh conditions. They grow well in low humidity, moist soil. Young plants can be grown from seeds. Petunias should be watered once every two to five days and in drier regions daily. Maximum growth occurs in late spring. Petunias are commonly cultivated, for example, in hanging baskets or in mixed in gardens.

Flower color is a complex trait involving pigments (mainly Flavonoids in the case of Petunia ), co-pigments, vacuolar pH, cell shape.

Petunia is an important and valuable bedding plant. Thus, the consumers are always looking for novelties.

The inventors of the present application have provided a Petunia flower having a novel phenotype.

### DETAILED DESCRIPTION

There is provided a Petunia flower having a blue or purple pigmentation, wherein said pigmentation is at a significantly higher concentration in the margin area of the flower compared with the center area of the flower and wherein the margin area and the center area of the flower are divided by a boundary.

In one embodiment, the pigmentation can be characterized according to the Royal Horticultural Society scale of values as being in the range 58 to 118, in particular in the ranges which specify blue color (N89 to 118) or purple color (58 to 88).

In one embodiment, a Petunia flower according to the present invention has a boundary between the margin area and the center area of the flower which is located such that the margin area may be expressed as a percentage of the total flower area.

In one embodiment, the margin area comprises not less than 5% of the total flower area, preferably not less than 10% of the total flower area, preferably not less than 20% of the total flower area, preferably not less than 50% of the total flower area when the flower is fully open.

In one embodiment, a Petunia flower according to the present invention has a boundary between the margin area and the center area of the flower which is located such that the margin area comprises less than 80% of the total flower area, preferably less than 60% of the total flower area when the flower is fully open.

In one embodiment, a Petunia flower according to the present invention has a pigmentation caused by a dominant mutation. In another embodiment, a Petunia flower according to the present invention has a pigmentation caused by a recessive mutation.

In one embodiment, there is provided a Petunia flower having a blue or purple margin according to the present invention obtainable from a Petunia plant P13-501, representative seed of which has been deposited at NCIMB under deposit number NCIMB 42187. This Petunia flower is representative of Pattern 1A of the present invention.

In one embodiment, there is provided a Petunia flower having a blue or purple margin according to the present invention obtainable from a Petunia plant P13-505, representative seed of which has been deposited at NCIMB under deposit number NCIMB 42188. This Petunia flower is representative of Pattern 1B of the present invention.

In one embodiment, there is provided a Petunia flower obtainable from a Petunia plant P12-2018, representative seed of which has been deposited at NCIMB under deposit number NCIMB 42189. This Petunia flower is representative of Pattern 2A of the present invention.

In one embodiment, there is provided a Petunia flower having a blue margin according to the present invention obtainable from a Petunia plant P12-2791, representative seed of which has been deposited at NCIMB under deposit number NCIMB 42190. This Petunia flower is representative of Pattern 2B of the present invention.

Pattern 1A and 1B are representative of Pattern 1 of the invention. Pattern 2A and 2B are representative of Pattern 2 of the invention.

In one embodiment, there is provided a Petunia flower according to the present invention wherein said flower has a pigment composition as measured by cinnamic acid derivative, flavonoid and anthocyanin profile which differs significantly from a flower with a morn pattern such as Merlin Blue Morn.

There is provided a Petunia flower according to the present invention, wherein one, most preferably two, of the following flavonoid compounds selected from the list of Kampferol and Quercitin may be detected in a sample of said flower.

There is provided a Petunia flower according to the present invention, wherein one, more preferably two, more preferably three, more preferably four, more preferably five, most preferably six of the anthocyanins selected from the list of Cyanidin, Pelargonidin, Petunidin, Malvidin, Delphinidin, Peonidin may be detected in a sample of said flower.

In one embodiment, the abundance of anthocyanins is lower in the center area of the flower compared with the margin area of the flower. In one embodiment, said lower abundance is present in Pattern 1. In one embodiment, said lower abundance is present in Pattern 2. In one embodiment, the abundance of anthocyanins in the center area of the flower is less than 20% of that found in the margin area of the flower.

In one embodiment, the abundance of flavonoids and the abundance of anthocyanins are lower in the center area of the flower compared to the margin area of the flower. In one embodiment, said lower abundance is present in Pattern 1. In one embodiment, said lower abundance is present in Pattern 2.

There is provided a Petunia flower according to the present invention, wherein one, most preferably two, of the following cinnamic acid derivatives selected from the list Caffeic acid, p-Coumaric acid may be detected in a sample of said flower.

In one embodiment, there is a higher abundance of cinnamic acid derivatives in the center area of the flower compared to the margin area of the flower. In one embodiment, said higher abundance is present in Pattern 1. In one embodiment, said higher abundance is present in Pattern 2

There is provided a Petunia flower according to the present invention wherein said flower has a gene expression profile in which one or more genes differs significantly from a flower with a morn pattern such as Merlin Blue Morn.

In one embodiment, the gene expression profile of the CHS genes is significantly different.

In one embodiment, an increased expression of the chalcone synthase (CHS) gene was observed in the margin area of the flower compared with expression of CHS gene in the center area of the flower. In one embodiment, said increased expression is present in Pattern 1. In one embodiment, said increased expression is present in Pattern 2.

In one embodiment, the relative expression of the CHS gene in the margin area of the flower compared with the center area of the flower is on average over 50,000 times greater. In one embodiment, the relative expression of the CHS gene in the margin area of the flower compared with the center area of the flower is on average over 100,000 times greater.

The flower of the invention is comprised of more than 1 genotype, and characterized as having either a sharp boundary between the margin area and center area or having a diffuse boundary. Values for the slope of color transition change can be analyzed by image analysis. Table 5 shows a comparison between the Morn, Pattern 1 and Pattern 2 type flowers.

A diffuse boundary can be characterized as having a low slope of transition change, for example in a Morn and Pattern 1 type flower. A sharp boundary can be characterized as having a high slope of transition change, for example in a Pattern 2 type flower.

In one embodiment, a flower of the invention has a slope of transition change value of over 20. In one embodiment, a flower of the invention has a slope of transition change value of over 30. In one embodiment, a flower of the invention has a slope of transition change value of over 35. In one embodiment, a flower of the invention has a slope of transition change value of between 20 and 40.

The center area of the flower of the invention is substantially non-pigmented and has a white appearance. The center area excludes the throat region. The throat region is of substantially different color from the surrounding center region.

In one embodiment, the flowering of the plant of the invention is early and continuous.

There is also provided a Petunia plant having a flower according to the present invention. In one embodiment, said plant is a hybrid. In one embodiment, said plant is an inbred line.

There is provided a Petunia plant according to the present invention, wherein said plant is tolerant to a pH value higher than 7 but less than 9, most preferably higher than 8 but less than 9 compared to a reference Petunia plant not having a flower according to the present invention.

In one embodiment, tolerance of a plant of the invention can be demonstrated in terms of a reduced level of leaf yellowing compared with a reference Petunia plant which is not tolerant to soils having pH value higher than 7 but less than 9.

In one embodiment, the plant of the invention is strong growing for fast and vigorous fill in baskets & containers.

In one embodiment, the plant of the invention has dark green foliage when grown in soils having pH values higher than 7 but less than 9.

In one embodiment, there is provided a Petunia plant having a flower according to the present invention, wherein said plant is grown under climatic conditions typical of a greenhouse.

In one embodiment, said climatic conditions are characterized by an average temperature during the day of 21° C.; an average temperature during the night of 18° C.; an average relative humidity of 72% over the 24 h period; and exposure of the plants to 14 h light per day.

There is also provided a plant part of a Petunia plant as herein described.

There is also provided seed or vegetative cutting of a Petunia plant as herein described.

There is also provided a method of producing a Petunia plant as herein described wherein the method comprises:

(a) crossing a first parent Petunia plant having a desired trait with a second parent Petunia plant to produce progeny plants; and (b) selecting one or more progeny plants that have the desired trait to produce selected progeny plants.

In one embodiment, there is provided a method according to the present invention, wherein the desired trait is tolerance to pH values higher than 7 but less than 9.

There is also provided seed of a Petunia plant produced by the method as herein described.

There is also provided a Petunia plant, or a part thereof, produced by growing the seed as herein described.

There is also provided a tissue culture of cells produced from a Petunia plant as herein described, wherein said cells of the tissue culture are produced from a plant part selected from the group consisting of seed, leaf, pollen, embryo, cotyledon, hypocotyl, meristematic cell, root, root tip, pistil, anther, flower, stem, and petiole.

There is also provided a Petunia plant regenerated from the tissue culture as herein described.

There is also provided a method for producing hybrid Petunia seed comprising crossing a first parent Petunia plant with a second parent Petunia plant and harvesting the resultant hybrid Petunia seed, wherein said first parent Petunia plant and/or second parent Petunia plant is a Petunia plant as herein described.

There is also provided a hybrid Petunia plant produced by growing hybrid Petunia seed as herein described.

There is also provided a Petunia flower having colored pigmentation in the margin area of the flower compared with the center area of the flower and wherein the margin area and the center area of the flower are divided by a boundary having a sharp boundary edge.

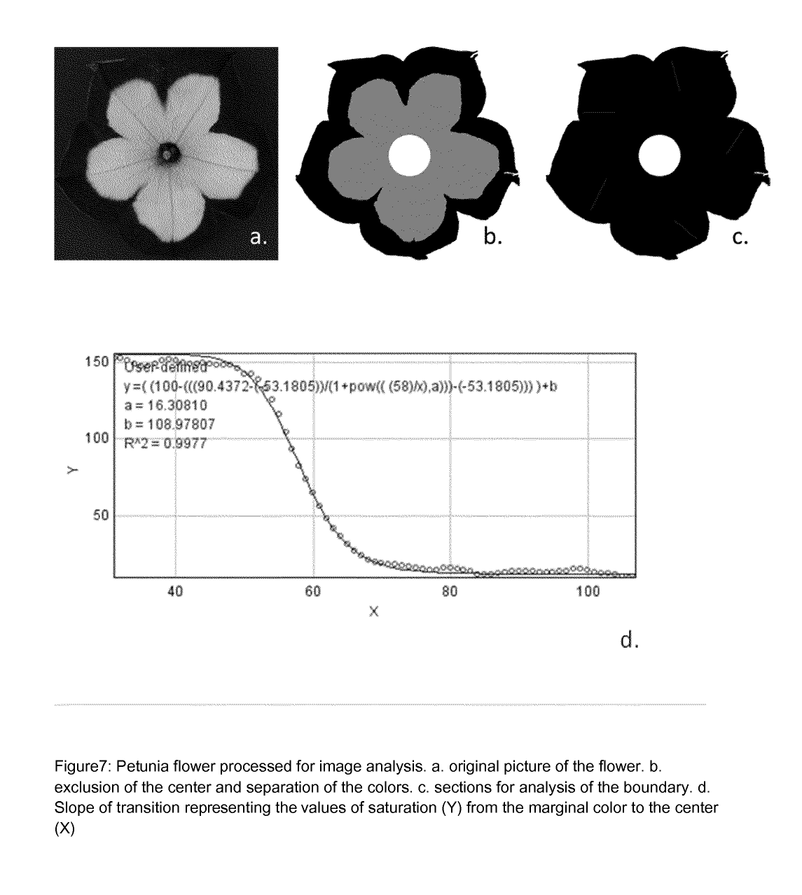

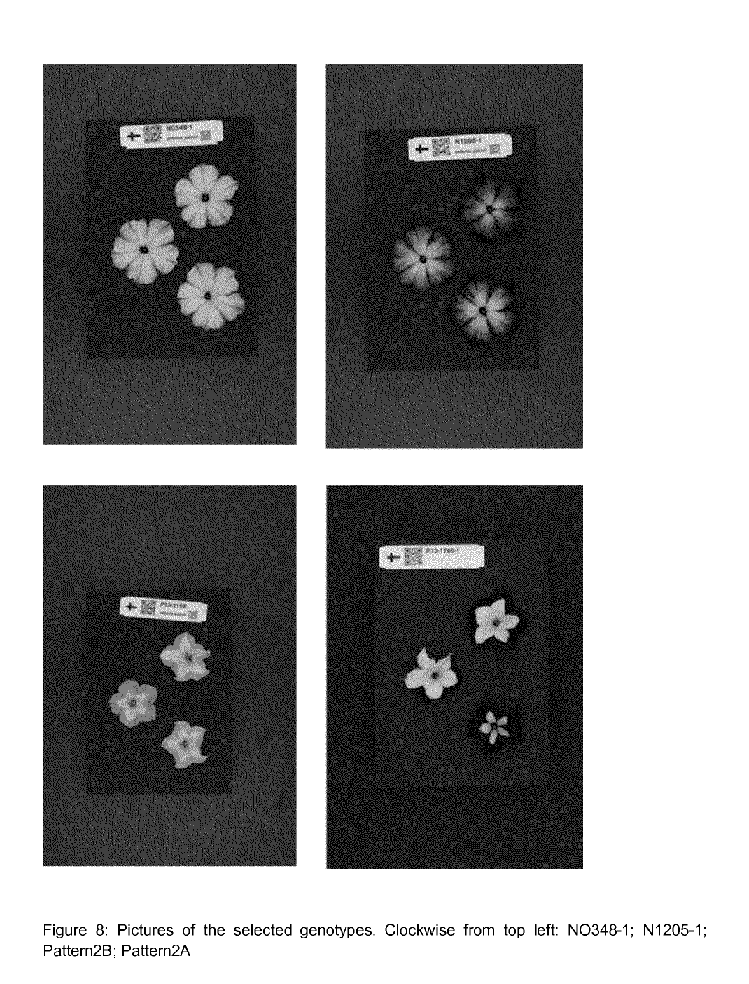

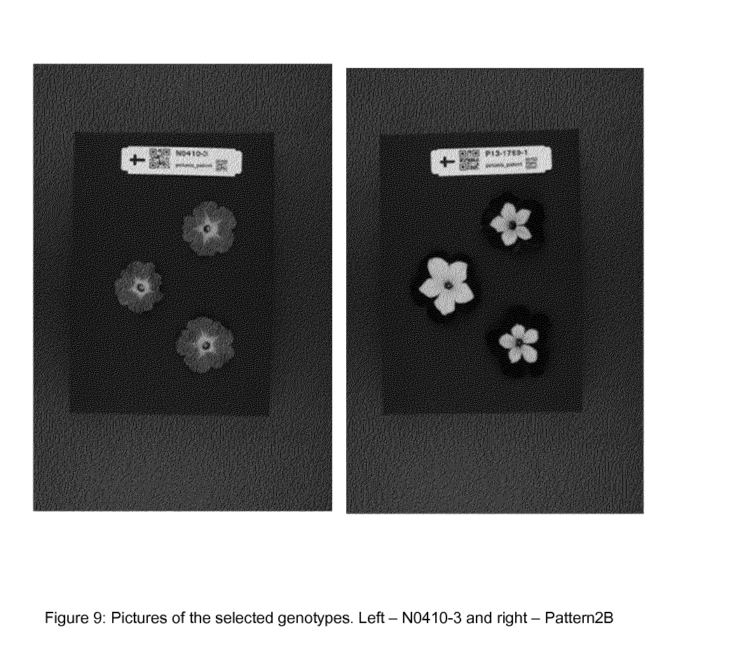

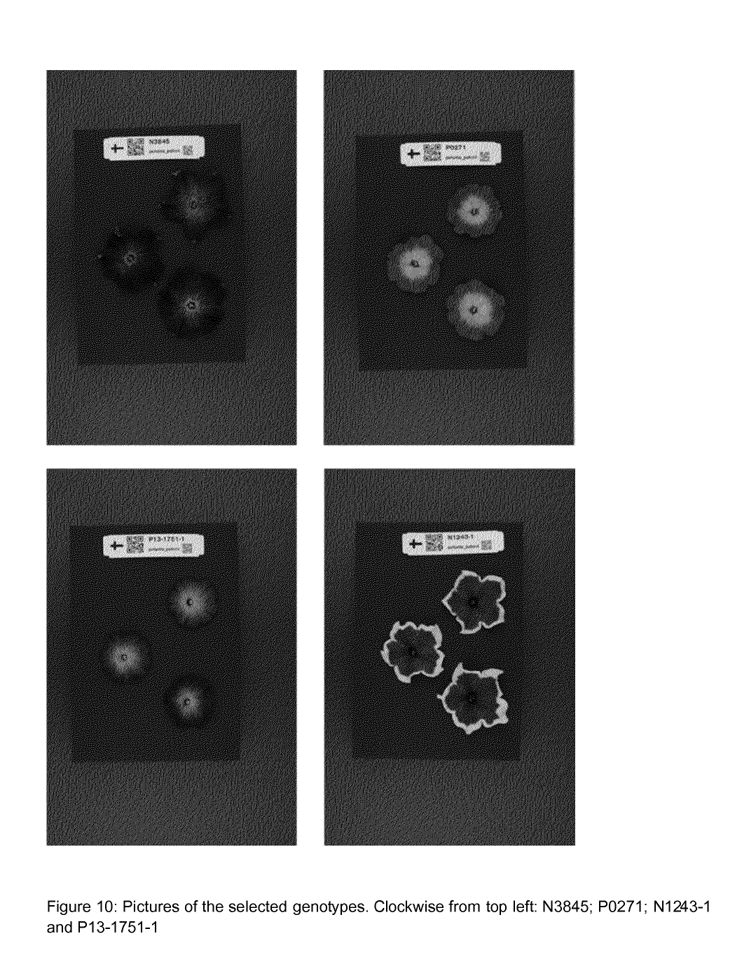

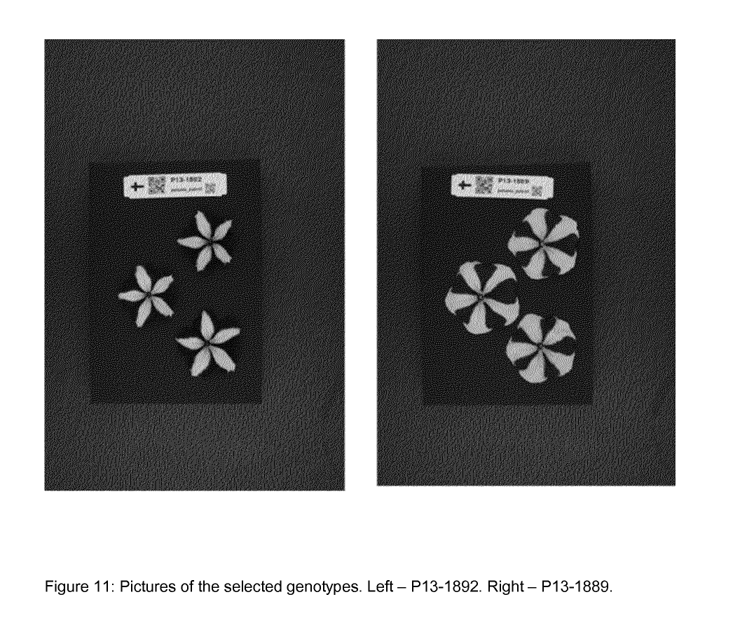

A typical example of such a flower is shown in FIG. 7a.

In one embodiment, said pigmentation is caused by a recessive mutation.

In one embodiment, said pigmentation is colored blue. In another embodiment, said pigmentation is colored purple. In another embodiment, said pigmentation is colored light pink, for example Petunia plant P12-2018. In another embodiment, said pigmentation is colored light lavender. In another embodiment, said pigmentation is colored rose. Alternatively, said pigmentation is selected from the following non-exhaustive list of colors: red, orange, yellow, green, indigo, and violet.

### DEFINITIONS

The technical terms and expressions used within the scope of this application are generally to be given the meaning commonly applied to them in the pertinent art of plant breeding and cultivation if not otherwise indicated herein below.

As used in this specification and the appended claims, the singular forms “a”, “an”, and “the” include plural referents unless the context clearly dictates otherwise. Thus, for example, reference to “a plant” includes one or more plants.

As used herein, the term “boundary” refers to the line separating the margin area of the flower from the center area of the flower. The boundary line forms an enclosed space around the center area of the flower. An example is illustrated in FIG. 7b.

As used herein, the term “breeding”, and grammatical variants thereof, refer to any process that generates a progeny individual. Breeding can be sexual or asexual, or any combination thereof. Exemplary non-limiting types of breeding include crossings, selfing, doubled haploid derivative generation, polyploidization and combinations thereof. The phenotype of the flower of the present invention can be readily and stably transferred by breeding to progeny.

As used herein, the term “hybrid” in the context of plant breeding refers to a plant that is the offspring of genetically dissimilar parents produced by crossing plants of different lines or breeds or species, including but not limited to the cross between two inbred lines.

A “plant” is any plant at any stage of development.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, the phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “progeny” refers to the descendant(s) of a particular cross. Typically, progeny result from breeding of two individuals, although some species (particularly some plants and hermaphroditic animals) can be selfed (i.e. the same plant acts as the donor of both male and female gametes). The descendant(s) can be, for example, of the F1, the F2, or any subsequent generation.

“Trait” is understood within the scope of the invention to refer to a characteristic or phenotype, for example blue or purple pigmentation or color. A trait may be inherited in a dominant or recessive manner, or may be monogenic or polygenic.

“Dominant” is understood within the scope of the invention to refer to an allele which determines the phenotype when present in the heterozygous or homozygous state.

A “recessive” allele is only displayed when present in the homozygous state.

“Throat” is understood within the scope of the invention to refer to the opening of a tubular corolla or calyx where the tube joins the limb. The petal limb is the spreading upper part of a tube-shaped flower “Margin” is understood within the scope of the invention to refer to the region of the flower which extends substantially around the circumference of the flower.

As used herein, the term “Center” area of the flower refers to the area which is enclosed by the margin, but which excludes the throat. When used in the context of the present invention, the center area is light colored when compared to the margin area.

### Seed Deposit Details

Seed of the variety P13-501 (a Petunia hybrida plant) has been deposited under the terms of the Budapest Treaty on 11^{th} Nov. 2013 at the NCIMB, Craibstone, Aberdeen, UK under number NCIMB 42187.

Seed of the variety P13-505 (a Petunia hybrida plant) has been deposited under the terms of the Budapest Treaty on 11^{th} Nov. 2013 at the NCIMB, Craibstone, Aberdeen, UK under number NCIMB 42188.

Seed of the variety P12-2018 (a Petunia hybrida plant) has been deposited under the terms of the Budapest Treaty on 11^{th} Nov. 2013 at the NCIMB, Craibstone, Aberdeen, UK under number NCIMB 42189.

Seed of the variety P12-2791 (a Petunia hybrida plant) has been deposited under the terms of the Budapest Treaty on 11^{th} Nov. 2013 at the NCIMB, Craibstone, Aberdeen, UK under number NCIMB 42190.

All seed deposits were made in the name of Syngenta Participations AG, Basel 4002, Switzerland.

### EXAMPLES

### Example 1

### Pigment Analysis in Floral Pattern—Selection and Sampling of Genotypes

The goal of this study was to characterize the novel reversed pattern in Petunia of the present invention in comparison with known existing patterns. A palette of different flower patterns present on the market was selected. Flavonoids and anthocyanins are the main pigments responsible for flower color in Petunia . The study focused on analyzing those families of pigments as well as their precursors (the cinnamic acid derivatives).

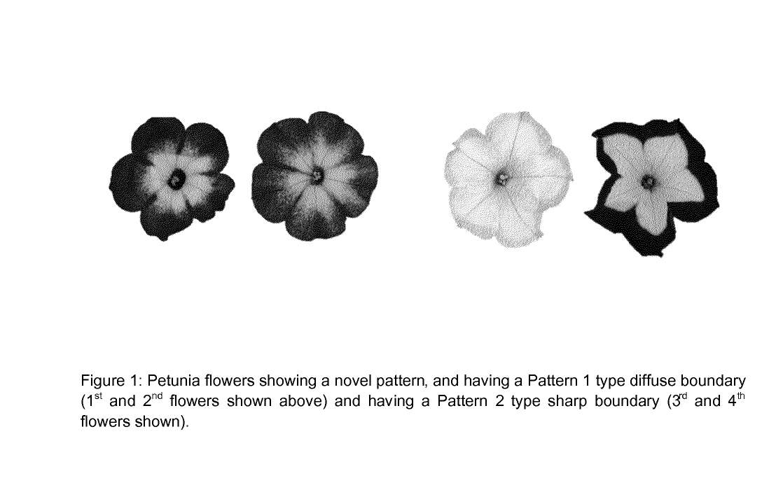

Material with a unique pattern has been created which shows a colored part on the margin area of the petal and a white center (see FIG. 1).

Eighteen petunia genotypes were selected for their flower color and pattern (see Table 1). Several patterns were investigated:
Pattern 1: marginal pattern with colored margin and light/white center delimited by a soft boundary edge (gradual transition for color)
Pattern 2: marginal pattern with colored margin and light/white center delimited by a sharp boundary edge
Morn: marginal pattern with colored margin and light/white center delimited by a soft boundary edge (gradual transition for color)
Picotee: marginal pattern with white margin and colored center delimited by a sharp boundary edge
Star: bicolor pattern forming a star white star on a colored petal with a sharp boundary edge.

Petunia plants were grown from cuttings until flowering.

TABLE 1
list of the selected genotypes.
Pattern, color & variety are reported as shown.

| ADMNCD | Type | Color |
|---|---|---|
| Pattern1A | Pattern1 | purple |
| Pattern1B | Pattern1 | blue |
| Pattern2A | Pattern2 | light pink |
| Pattern2B_1 | Pattern2 | blue |
| Pattern2B_2 | Pattern2 | blue |
| N0410-3 | Synguna Compact Pink Morn | pink |
| N3845 | Sophistica Blue Morn | blue |
| P0271 | Fantasy Hot Pink Morn | rose |
| P13-1751-1 | Merlin Blue Morn | blue |
| N1243-1 | Synguna Purple Picotee | purple |
| P13-1892 | Star | blue |
| P13-1889 | Star | purple |

### Sampling

Flowers were collected at a freshly open flower stage in a cool box before dissection. Up to 5 flowers were used to collect sufficient amount of tissue. Colored and light/white part of the petals were separated with tweezers and scalpels (throat and reproductive organs were removed). Dissected petals were collected per color in 50 mL tubes, freeze dried overnight and stored at −80 C. until shipping.

Samples were shipped under cool conditions for pigment extraction and analysis.

### Example 2

### Pigment Analysis in Floral Pattern—Extraction and Analysis of Pigments

Samples were processed using fast prep. Fine anhydrous powder formed with all samples
1. Petal homogenate (˜50 mg) was transferred to a cryovial.
2. A small amount of garnet matrix was added to the tube along with a ceramic ball.
3. Deionised water (1 ml) was added to the tube and the contents shaken for 1 minute on a shaker mill.
4. Ethyl acetate (2.5 ml) was then added to the cryovial and the tube shaken on a shaker mill for 5 minutes.
5. The resulting suspension was then centrifuged at 4000 rpm at 4° C. for 10 minutes.
6. The ethyl acetate layer was removed and discarded.
7. The remaining pellet and aqueous layer were then extracted with 2×2.5 ml MeOH/H₂O /HCOOH (5:5:1) as described above.

The two resulting polar extracts were combined and stored frozen at −20° C. to await HPLC-MS/MS analysis.

A high performance liquid chromatography-high resolution mass spectrometry (HPLC-HRMS) method was used for screening of the pigments.

### Example 3

### Pigment Analysis in Floral Pattern—Results of Analysis

The compounds detected among all the samples are listed in table 2.

TABLE 2
Pigment aglycones detected in the pigment analysis.

| Category | Compound | Formula |
|---|---|---|
| Flavonoids | Kampferol | C₁₅H₁₀O₆ |
| | Quercitin | C₁₅H₁₀O₇ |
| Anthocyanins | Cyanidin | C₁₅H₁₁O₆ |
| | Pelargonidin | C₁₅H₁₁O₅ |
| | Petunidin | C₁₆H₁₃O₇ |
| | Malvidin | C₁₇H₁₅O₇ |
| | Delphinidin | C₁₅H₁₁O₇ |
| | Peonidin | C₁₆H₁₃O₆ |
| Cinnamic derivaties | Caffeic acid | C₉H₈O₄ |
| | p-Coumaric acid | C₉H₈O₃ |

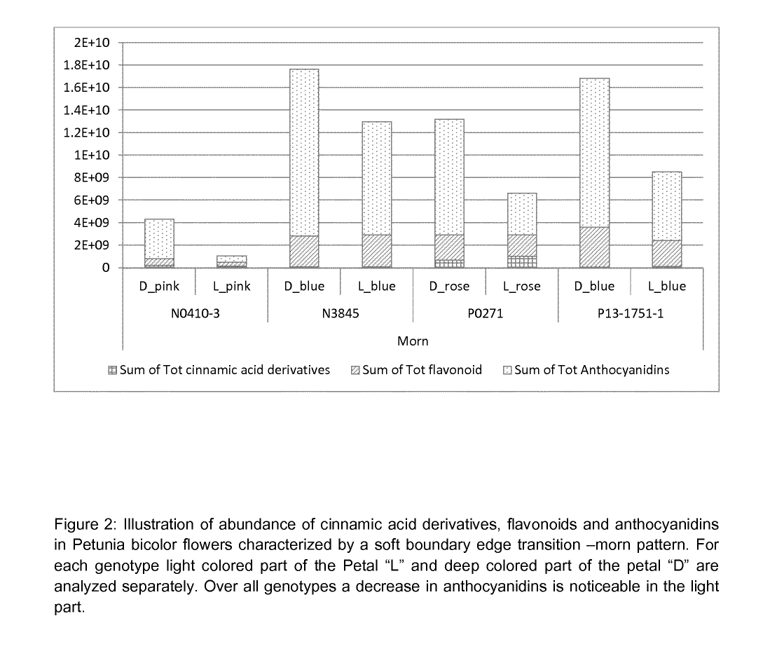

Among all genotypes with a morn pattern a decrease in accumulation of anthocyanidins is observed in the light part versus the dark colored part of the petals (see FIG. 2).

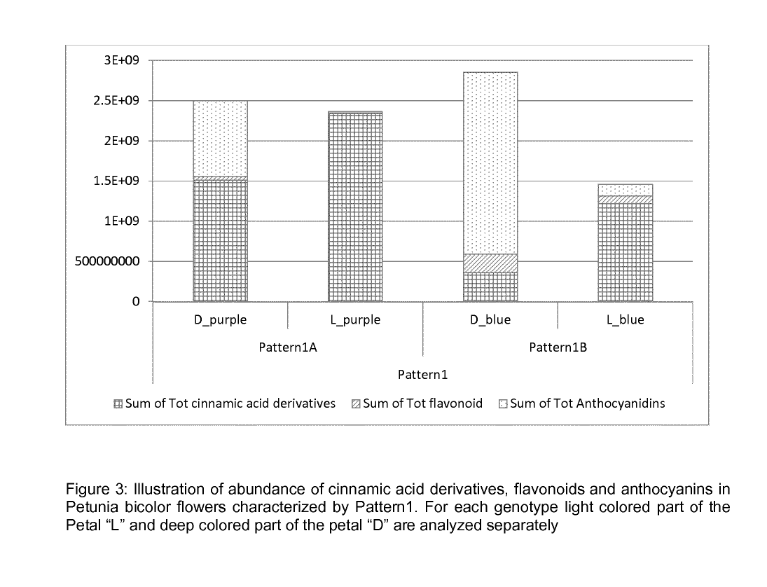

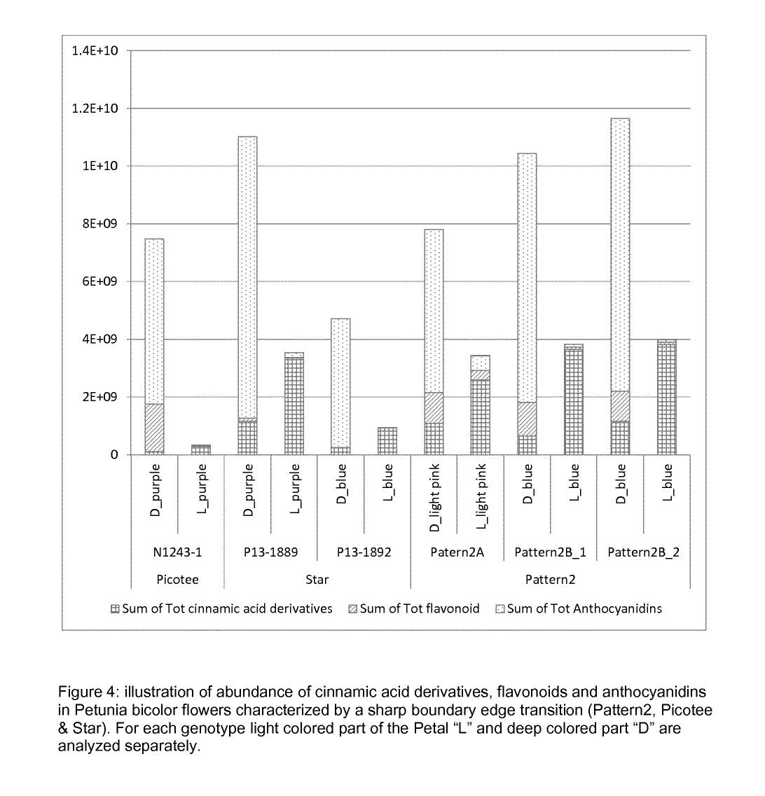

Pattern 1 is characterized by higher abundance of cinnamic acid derivatives in the light part compare to the dark part. Additionally, flavonoids and anthocyanins are highly decreased or absent in the light colored tissue (see FIG. 3).

Sharp boundary edge patterns have an increased abundance of cinnamic acid derivatives in the light part compared to the colored part. At the opposite, abundance of flavonoids and anthocyanins is reduced/absent in this same tissue

When compared to all other genotypes, the concentration of flavonoids & anthocyanins in the light part of the genotype Pattern 2A (Pattern 2) is quite high.

The pattern 1&2 show a reduction/absence of flavonoid and anthocyanins in the light part versus the colored part, at the opposite the concentration in cinnamic acid derivatives is increased in the light tissue.

In our new patterns 1&2 significant levels of anthocyanins and flavonoids are only found in the deeply colored area of the petal. Those results are consistent with the gene expression study previously reported. In both patterns a clear reduction of expression of chalcone synthase (Chs) gene was observed in the light part of the petals. CHS is known to encode an enzyme responsible for transformation of cinnamic acids into flavonoids.

### Example 4

Gene Expression Analysis—Sampling and qRT-PCR Conditions

A gene expression study was performed on the “light” and “colored” part of the flowers and compared to known existing Petunia patterns.

The goal of this study was to characterize the novel reversed pattern in Petunia of the present invention in comparison with existing ones. A palette of different flower patterns present on the market has been selected as described in Example 1. Knowing that flavonoids and anthocyanins are the main pigments responsible for flower color in Petunia , this investigation focused on analyzing transcripts of genes involved in the anthocyanin pathway.

Eighteen petunia genotypes were selected for their flower color and pattern and several patterns were sampled as described above in Example 1 and Table 1.

Dissected petals were collected per color in 50 mL vials. Once filled, vials were immediately frozen in liquid nitrogen and then stored at −80 C. until shipping. Samples were shipped to the United States for RNAs extraction and qRT-PCR assays on dry ice.

RNAs preparation was performed manually with the RiboPure extraction kit. The RNA adsorbs to the silica membrane in the presence of high concentrations of salt. Contaminants are unable to bind to the silica column and therefore pass through the column. The loaded column is washed and then any bound RNA is eluted.

Primer sets used for RT-PCR amplification were previously published (see Table 3). Tubulin assay was used as a reference for calculating a relative expression value.

TABLE 3
Primer pairs for real time PCR

| Gene | | Primer sequence (5′- 3′) | Remark |
|---|---|---|---|
| Chalcone synthase | GB0964 | 5′-GAACAGCCACACCTACAAAC-3′ | Griesbach, Beck & Hammond 2007 |
| gene (Chs) | | 5′-AACCCTGCTGGTACATCATG-3′ | |
| Tubulin (Tub) | GB0969 | 5′-TAGCGAAACCAGTGCTGGAAAG-3′ | Griesbach, Beck & Hammond 2007 |
| | | 5′-GCTTGAGGGCTCAAAAACAG-3′ | |

### Amplification:

10 cycles of 94° C. for 30 s, 55° C. for 30 s, 68° C. for 60 s followed by 25 cycles of 94° C. for 30 s, 55° C. for 30 s, and 68° C. for 60 s. (Griesbach et al 2007, J. AMER. SOC. HORT. SCI. 132(5), 680-690.)

### Example 5

### Gene Expression Analysis—Results of Analysis

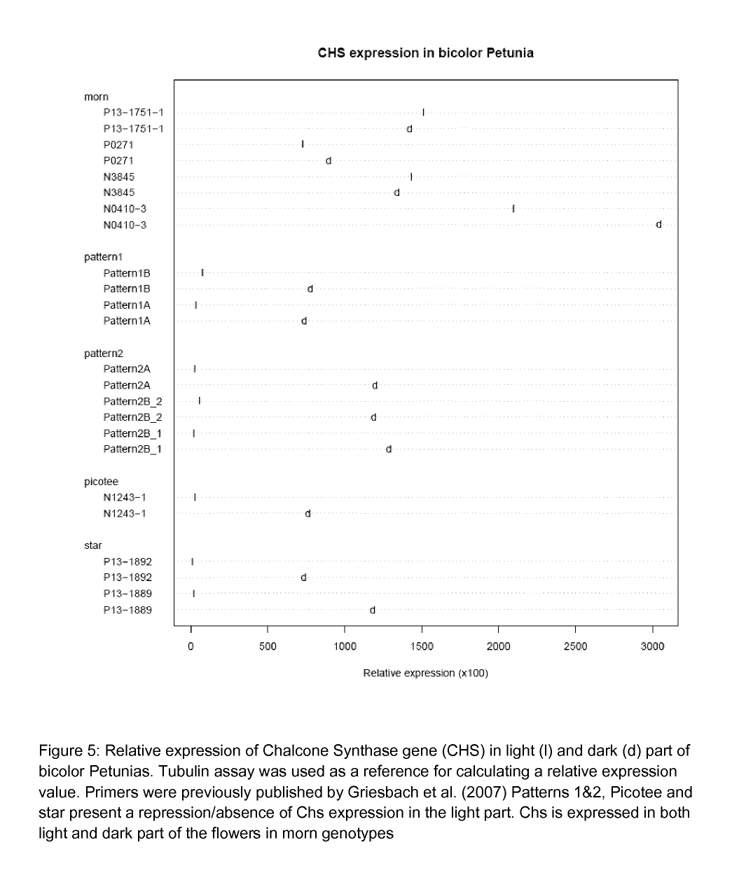

Calculated expression value is presented in FIG. 5 per type of pattern.

The focus of this experiment was on the expression pattern (on/off) of the Chs gene involved in the anthocyanin pathway. Chs expression was evaluated using oligos from Griesbach et al. The level of Chs expression was significantly reduced in the light part of petals in the Picotee and Star patterns. This was previously reported by Griesbach et al. Morn patterns did not show difference in Chs expression between the two colored tissues. Patterns 1 & 2 had similar expression pattern as Star and Picotee for Chs.

This study suggests that repression of Chalcone synthase (Chs) in the “light” part of both of the novel patterns (Pattern 1 & Pattern 2) is involved in the formation of the highly white part of the new genotypes.

### Example 6

### Image Analysis—Flower Color

Flowers were freshly picked at open flower stage and placed on a support so they would face up and the corolla would be as flat as possible. Pictures were taken with a camera Canon EOS 550D mounted on a fix arm. The setup was in a dark room with artificial lights only allowing reproducible conditions.

Prior to any flower shooting, a picture of a X-Rite color was made. This enables to get accurate color profiles of the flowers. 6 flowers per genotype were analyzed.

Flower color was calculated using ImageJ. For each flower a sample dark and light color were selected manually for each color. The median value was used to characterize respectively the “dark”and “light”color.

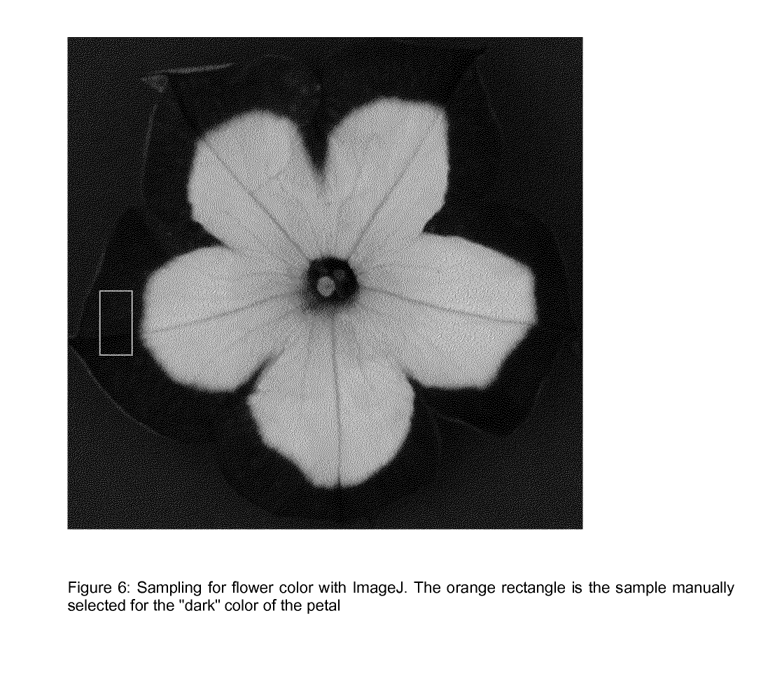

An example of measurement is presented FIG. 6.

TABLE 4
Average measurements of flower color via image analysis.
6 flowers per genotype were analyzed.

| | | | | light part | | |
|---|---|---|---|---|---|---|
| | dark part | | | | | Bright- |
| | Hue | Saturation | Brightness | Hue | Saturation | ness |
| morn | | | | | | |
| N0410-3 | 233 | 234 | 162 | 16 | 52 | 162 |
| N3845 | 185 | 237 | 46 | 89 | 54 | 79 |
| P0271 | 237 | 254 | 148 | 45 | 67 | 166 |
| P13-1751-1 | 188 | 220 | 93 | 209 | 35 | 134 |
| pattern1 | | | | | | |
| Pattern1A | 223 | 205 | 147 | 19 | 15 | 182 |
| Pattern1B | 195 | 238 | 59 | 60 | 24 | 163 |
| pattern2 | | | | | | |
| Pattern2A | 198 | 68 | 160 | 39 | 20 | 179 |
| Pattern2B_1 | 182 | 234 | 41 | 44 | 20 | 180 |
| Pattern2B_2 | 182 | 240 | 42 | 45 | 36 | 180 |
| picotee | | | | | | |
| N1243-1 | 220 | 251 | 90 | 31 | 10 | 171 |
| star | | | | | | |
| P13-1889 | 236 | 252 | 64 | 34 | 25 | 180 |
| P13-1892 | 198 | 229 | 41 | 33 | 15 | 181 |

### Example 7

### Image Analysis—Color Transition

Calculated color transition is presented in FIG. 7.

Flowers were converted to black and white pictures. Sections were drawn manually across the transition. Five sections per flower were made and 6 flowers were analyzed per genotype. The saturation value of the each pixel of this section plotted on a graph. The curved was analyzed and the slope of the curve use as reference for flower color transition. Values are presented in the table 5 below.

Slope of color transition of Morn genotypes and Pattern 1 genotypes do no show significant differences. Low values are characteristic of soft boundary edge pattern with a gradual transition from dark to light color. Pattern 2B show significantly higher slope of transition characteristic to the sharp boundary edge pattern.

1. A Petunia flower having a blue or purple pigmentation, and wherein said pigmentation is at a significantly higher concentration in the margin area of the flower compared with the center area of the flower and wherein the margin area and the center area of the flower are divided by a boundary.

2. A Petunia flower according to claim 1, wherein the pigmentation can be characterized according to the Royal Horticultural Society scale of values as being in the range 58 to 118.

3. A Petunia flower according to claim 1, wherein the boundary between the margin area and the center area of the flower which is located such that the margin area may be expressed as a percentage of the total flower area.

4. A Petunia flower according to claim 1, wherein the margin area comprises not less than 5% of the total flower area when the flower is fully open.

5. A Petunia flower according to claim 1, wherein the margin area comprises less than 80% of the total flower area when the flower is fully open.

6. A Petunia flower according to claim 1, obtainable from a Petunia plant P13-501, representative seed of which has been deposited at NCIMB under deposit number NCIMB 42187.

7. A Petunia flower according to claim 1, obtainable from a Petunia plant P13-505, representative seed of which has been deposited at NCIMB under deposit number NCIMB 42188.

8. A Petunia flower according to claim 1, obtainable from a Petunia plant P12-2018, representative seed of which has been deposited at NCIMB under deposit number NCIMB 42189.

9. A Petunia flower according to claim 1, obtainable from a Petunia plant P12-2791, representative seed of which has been deposited at NCIMB under deposit number NCIMB 42190.

10. A Petunia flower according to claim 1, wherein said flower has a pigment composition as measured by cinnamic acid derivative, flavonoid and anthocyanin profile which differs significantly from a flower with the morn pattern of Merlin Blue Morn.

11. A Petunia flower according to claim 1, wherein at least one of the flavonoid compounds selected from the list of Kampferol and Quercitin may be detected in a sample of said flower.

12. A Petunia flower according to claim 1, wherein at least one of the anthocyanins selected from the list of Cyanidin, Pelargonidin, Petunidin, Malvidin, Delphinidin, Peonidin may be detected in a sample of said flower.

13. A Petunia flower according to claim 1, wherein the abundance of anthocyanins is lower in the center area of the flower compared with the margin area of the flower.

14. A Petunia flower according to claim 1, wherein the abundance of anthocyanins in the center area of the flower is less than 20% of that found in the margin area of the flower.

15. A Petunia flower according to claim 1, wherein the abundance of flavonoids and the abundance of anthocyanins are lower in the center area of the flower compared to the margin area of the flower.

16. A Petunia flower according to claim 1, wherein said flower has a gene expression profile which differs significantly from a flower with the morn pattern of Merlin Blue Morn.

17. A Petunia flower according to claim 1, wherein an increased expression of the chalcone synthase (CHS) gene was observed in the margin area of the flower compared with expression of CHS gene in the center area of the flower.

18. A Petunia flower according to claim 1, wherein the boundary is characterized as having a slope of transition change value of over 20.

19. A Petunia plant having a flower according to claim 1, wherein said plant is a hybrid or an inbred line.

20. A Petunia plant according to claim 19, wherein said Petunia plant is tolerant to a pH value higher than 7 compared to a Petunia plant having a flower which does not contain said blue or purple pigmentation.

21. A Petunia plant according to claim 19, wherein said plant is grown under climatic conditions typical of a greenhouse.

22. A Petunia plant according to claim 21, wherein said climatic conditions are characterized by an average temperature during the day of 21° C.; an average temperature during the night of 18° C.

23. Plant part of a Petunia plant according to claim 19.

24. Seed or vegetative cutting of a Petunia plant according to claim 19.

25. A method of producing a Petunia plant according to claim 19 wherein the method comprises:
(a) crossing a first parent Petunia plant having a desired trait with a second parent Petunia plant to produce progeny plants; and
(b) selecting one or more progeny plants that have the desired trait to produce selected progeny plants.

26. The method according to claim 25, wherein the desired trait is tolerance to pH values higher than 7.

27. Seed of a Petunia plant produced by the method of claim 25.

28. A Petunia plant, or a part thereof, produced by growing the seed of claim 27.

29. A tissue culture of cells produced from the Petunia plant of claim 28, wherein said cells of the tissue culture are produced from a plant part selected from the group consisting of seed, leaf, pollen, embryo, cotyledon, hypocotyl, meristematic cell, root, root tip, pistil, anther, flower, stem, and petiole.

30. A Petunia plant regenerated from the tissue culture of claim 29.

31. A method for producing hybrid Petunia seed comprising crossing a first parent Petunia plant with a second parent Petunia plant and harvesting the resultant hybrid Petunia seed, wherein said first parent Petunia plant and/or second parent Petunia plant is the Petunia plant of claim 19.

32. A hybrid Petunia plant produced by growing said hybrid Petunia seed of claim 31.
